(19) United States
(12) Patent Application Publication
(10) Pub. No.: US 2026/0224517 A1
(43) Pub. Date: Aug. 6, 2026

(54) AN IMMEDIATE-RELEASE TABLET OF PREGABALIN WITH AN INCREASED PERCENTAGE API CONTENT

(71) Applicant: Lasse KINAST, Berlin (DE)

(72) Inventor: Lasse KINAST, Berlin (DE)

(21) Appl. No.: 19/152,887

(22) PCT Filed: Feb. 7, 2024
(86) PCT No.: PCT/EP2024/053053

§ 371 Date: Jul. 31, 2025

(30) Foreign Application Priority Data
Feb. 7, 2023 (DE) 10 2023 000 369.7

(51) Int. Cl. A61K 31/197; A61K 9/20

(52) U.S. Cl. CPC A61K 31/197; A61K 9/2027; A61K 9/2054

(57) ABSTRACT

The invention provides an immediate-release oral pharmaceutical composition, e.g. a tablet which comprises by weight ≥50.1% Pregabalin, ≥8.0% polyvinylpyrrolidone, ≥3.0% croscarmellose sodium, and from 0% to 38.9% additional pharmaceutical excipients.

The invention provides the composition of an immediate-release tablet which comprises by weight ≥50.1% Pregabalin (at a median particle size of less than 200 μm), ≥8.0% polyvinylpyrrolidone, ≥3.0% croscarmellose sodium, and from 0% to 38.9% additional pharmaceutical excipients. With the composition invented, the prepared Pregabalin immediate-release tablet has the benefit of a higher percentage API content while also having a surprisingly high tablet hardness. The Pregabalin entails also the other pharmaceutically acceptable salts of Pregabalin.

### TECHNICAL FIELD

The invention belongs to the field of pharmaceutical preparations, a composition of matter, for the composition of a Pregabalin, or racemate thereof, immediate-release tablet.

### BACKGROUND

Pregabalin is an anticonvulsant, analgesic and anxiolytic medication used to treat epilepsy, neuropathic pain, fibromyalgia, restless leg syndrome, opioid withdrawal and generalized anxiety disorder (GAD).

Pregabalin is typically administered in the form of an immediate-release capsule, a controlled release tablet and rarely also as an immediate-release tablet. A typical Pregabalin immediate-release tablet on the market contains roughly 40% Pregabalin by weight and a typical immediate-release capsule contains roughly 60% Pregabalin by weight. The standard dosages being from 25 mg to 300 mg. Pregabalin dosage forms are usually and out of necessity packaged in PVC/ALU or ALU/ALU blisters, to ensure the stability of the product.

The environmental burden of pharmaceutical packaging is immense. This is because the aluminium and plastics used to form the blister packaging are difficult and even impossible to recycle effectively as the materials are sealed together. Separating the materials again requires high amounts of energy and even then, the aluminium received from this is not of the same high quality as new aluminium. The larger the immediate-release oral pharmaceutical dosage form is, the larger the primary packaging must be and therefore also the more waste material is created.

Additionally, there is the environmental burden caused through the extensive transportation steps required of the finished product, such as the transportation from the manufacturer to the wholesaler and then to the pharmacy. Each box of finished product in the pharmaceutical industry needs to be transported several steps until it reaches the customer, often even global transportation routes are involved. This is especially the case for generic medicines, and further relevant for products which are sold in large quantities, like with Pregabalin. The volume of the finished product determines how efficiently transportation and storage can be conducted, since a product with a high volume will require more space and more shipments compared to the same product at a smaller volume.

Furthermore, when it is avoidable to expose the patient to excipients, it should be done. An immediate-release tablet is administered with the goal of subjecting the human body to the drug and not the excipients. It is a waste of resources of the pharma industry to use excipients in products where they can be avoided, especially when used in high quantities. Also, the global demand of excipients is ever increasing, and the supply capabilities of the chemical industry is falling behind the demand which makes this waste of recourses even more problematic.

Previous patented combinations of Pregabalin tablets with an increased API content above 50%, are mostly for controlled-release (sustained-release or extended-release) formulations and not for immediate-release tablets. Such is the case with AU2017300185 (65% Pregabalin content by weight) and WO2016187718 (95% Pregabalin content by weight). There has been research done and patents made for Pregabalin immediate-release tablets, but under the condition that very specific excipients must be used for tabletting success. Such is the case with WO2008128775 which requires the use of an alkaline earth phosphate or pentite/hexite or polyacrylate or surface-active substance and with WO2011107812A2 which requires the use of a disaccharide or higher polyol as stabilizer. Furthermore, the patent CN103494796A discloses the formulation of an immediate-release tablet, with a high percentage Pregabalin, but under the condition that Pregabalin at a median particle size of >200 μm must be used.

Pregabalin at a particle size <200 μm exhibits a lack of tabletability, due to a shortcoming in tablet hardness once pressed. This may be a leading reason why Pregabalin is typically manufactured in capsule form and not tablet form. Adding binder excipients to the pregabalin powder did not improve the tabletability enough for further processing.

We surprisingly found a very specific composition in which Pregabalin can be processed into tablets with sufficient hardness that allows for further handling and processing. This discovery allows for pregabalin tablets to be manufactured at higher percentage API content, from a particle size of <200 μm and this composition is not reliant on previously believed necessary excipients required to make up a stable composition.

Currently, on the market Pregabalin 25 mg immediate-release tablets weigh roughly 64 mg, and Pregabalin 300 mg tablets weigh roughly 762 mg. The excipient content is 39 mg and 362 mg per tablet respectively. The 25 mg immediate-release Pregabalin capsules weigh roughly 69 mg and the 300 mg immediate-release Pregabalin capsules weigh roughly 489 mg. The excipient content (including the capsule weight) is 24 mg and 189 mg respectively. Through this invention the excipient content can be brough down to only 11%, in the form of a tablet, or roughly 3 mg for the 25 mg tablet and 37 mg for the 300 mg tablet.

As the amount of excipient needed is reduced to a minimum, the immediate-release tablet shrinks, which in turn shrinks the size of the blister and shrinks the size of the entire finished product. Now a much smaller sized blister can be used, and this enables the finished product to have a fraction of the current volume. This in turn allows for much more efficient transportation, lowering the carbon footprint of the product, and only a fraction of the packaging waste is created.

It also reduces the amount of excipient administered to the patient down to the indispensable and drastically alleviates the pressure on the pharmaceutical industry to seek out and process an unnecessarily high quantity of excipient.

As Pregabalin is a high-volume prescription medication, it is synthesized in many production plants and the finished product is manufactured globally. The facilities and equipment needed to implement production of the given combination is readily available. The following examples of composition and preparation can realistically be implemented at any production site intended for tabletting.

### BRIEF DESCRIPTION OF THE FIGURES

FIG. 1 shows a graph representing exemplary results of the dissolution study in the below examples.

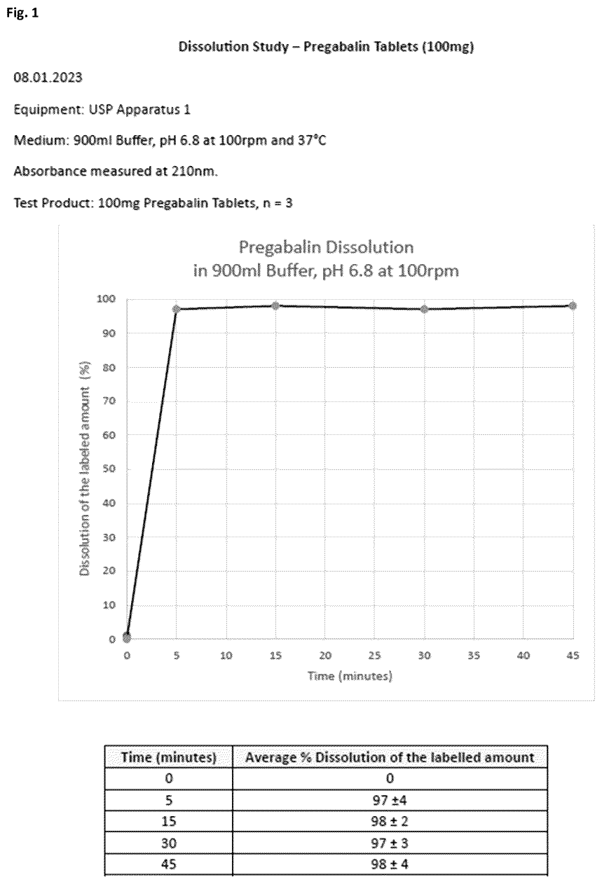

### EXAMPLES

### Example 1—Example Preparation of Pregabalin Film-Coated Tablets

Step 1. Pregabalin, polyvinylpyrrolidone and croscarmellose sodium are blended together. Pregabalin, polyvinylpyrrolidone and croscarmellose sodium may be passed through an appropriate screen before blending. For the avoidance of doubt, a screen in this context refers to a sieve, preferably sieves with a pore size of 250 μm-710 μm.
Step 2. Optional additional excipients are added and blended. Additional excipients may be passed through an appropriate screen (see above) before adding.
Step 3. The final blend is compressed into tablets.
Step 4. A film-coating can be added onto the tablets.

The methods of preparation may include but are not limited to the direct compression and wet granulation methods. The direct compression method is preferred, and an exemplary compression method is outlined in [16]. The compression force in the direct compression method in particularly is 40 kg/mm² or higher, more particularly 45 kg/mm² or higher, more particularly 50 kg/mm² or higher, more particularly 55 kg/mm² or higher, more particularly 60 kg/mm² or higher, more particularly 65 kg/mm² or higher, more particularly 70 kg/mm² or higher, and in any of the aforementioned embodiments even more particularly up to 75 kg/mm².

### Example Compositions

TABLE 1
Composition Example 1

| Component | Composition (%) |
|---|---|
| Pregabalin | 89.0 |
| Croscarmellose Sodium | 3.0 |
| Polyvinylpyrrolidone | 8.0 |
| Total | 100.0 |

TABLE 2
Composition Example 2

| Component | Composition (%) |
|---|---|
| Pregabalin | 87.0 |
| Croscarmellose Sodium | 3.0 |
| Polyvinylpyrrolidone | 8.0 |
| Talc | 1.0 |
| Magnesium Stearate | 1.0 |
| Total | 100.0 |

TABLE 3
Composition Example 3

| Component | Composition (%) |
|---|---|
| Pregabalin | 50.1 |
| Croscarmellose Sodium | 30.0 |
| Polyvinylpyrrolidone | 19.9 |
| Total | 100.0 |

TABLE 4
Composition Example 4

| Component | Composition (%) |
|---|---|
| Pregabalin | 50.1 |
| Croscarmellose Sodium | 27.0 |
| Polyvinylpyrrolidone | 19.9 |
| Film-coating | 3.0 |
| Total | 100.0 |

### Example 2—Example Tablet Weights form Different Compositions

TABLE 5
Overview of example tablet weights

| | | Example |
|---|---|---|
| Composition Example | Example Dosage | Total Weight |
| Composition Example 1 | 25 mg Tablet | 28.1 mg |
| Composition Example 1 | 600 mg Tablet | 674.2 mg |
| Composition Example 2 | 25 mg Tablet | 28.7 mg |
| Composition Example 2 | 300 mg Tablet | 344.8 mg |
| Composition Example 3 | 25 mg Tablet | 49.9 mg |
| Composition Example 4 | 300 mg Tablet | 598.8 mg |

### Example 3—Analytical Results

TABLE 6
Disintegration Time & Resistance to Crushing

| Example | Disintegration | Resistance to |
|---|---|---|
| Composition | Time (Minutes) | Crushing (N) |
| 1 | <1 min | >15N |
| 2 | <1 min | >15N |
| 3 | <1 min | >15N |
| 4 | <1 min | >15N |

Disintegration studies were conducted on tablets of Pregabalin according to apparatus A of the European Pharmacopeia, 2.9.1. Disintegration medium was 1 L of buffer at pH 6.8 at 37° C. Resistance to Crushing was tested according to Pharmacopeia 2.9.8 with a Dr. Schleuniger THP-4M 4M Tablet Hardness Tester.

A dissolution study was conducted on tablets of Example 3 in 900 ml of pH 6.8 buffer at 37° C., USP Apparatus 1 set to 100 rpm. The resultant dissolution curve demonstrates that the tablet rapidly dissolved with complete dissolution within 30 minutes.

### Example 4—Comparative Examples: Compositions which Didn't Meet the Threshold to Quality Requirements

TABLE 7
Composition Example 5

| Component | Composition (%) |
|---|---|
| Pregabalin | 90.0 |
| Polyvinylpyrrolidone | 10.0 |
| Total | 100.0 |

TABLE 8
Composition Example 6

| Component | Composition (%) |
|---|---|
| Pregabalin | 90.0 |
| Microcrystalline Cellulose | 10.0 |
| Total | 100.0 |

TABLE 9
Composition Example 7

| Component | Composition (%) |
|---|---|
| Pregabalin | 90.0 |
| Lactose | 10.0 |
| Total | 100.0 |

TABLE 10
Composition Example 8

| Component | Composition (%) |
|---|---|
| Pregabalin | 89.0 |
| Croscarmellose Sodium | 3.0 |
| Microcrystalline Cellulose | 8.0 |
| Total | 100.0 |

TABLE 11
Composition Example 9

| Component | Composition (%) |
|---|---|
| Pregabalin | 97.0 |
| Croscarmellose Sodium | 3.0 |
| Total | 100.0 |

TABLE 12
Composition Example 10

| Component | Composition (%) |
|---|---|
| Pregabalin | 92.0 |
| Polyvinylpyrrolidone | 8.0 |
| Total | 100.0 |

TABLE 13
Composition Example 11

| Component | Composition (%) |
|---|---|
| Pregabalin | 92.0 |
| Polyvinylpyrrolidone | 6.0 |
| Croscarmellose Sodium | 2.0 |
| Total | 100.0 |

TABLE 14
Composition Example 12

| Component | Composition (%) |
|---|---|
| Pregabalin | 94.0 |
| Polyvinylpyrrolidone | 4.5 |
| Croscarmellose Sodium | 1.5 |
| Total | 100.0 |

The ability to achieve tablets of an adequate hardness for further handling and processing is made possible only through the specific composition of the invention. Comparative compositions (see above) proved to have lower hardness than the compositions of the present invention, all exhibiting ≤15N resistance to crushing in the test as described herein above, making them less favourable for handling and processing.

Certain embodiments of the invention are enumerated in the following:
1. A pharmaceutical composition of an immediate-release tablet which comprises by weight of ≥50.1% Pregabalin, ≥8.0% polyvinylpyrrolidone, ≥3.0% croscarmellose sodium, and from 0% to 38.9% additional pharmaceutical excipients. More preferably it comprises by weigh of ≥70.1% Pregabalin and even more preferably it comprises by weight of ≥80.1% Pregabalin.
2. The Pregabalin of claim 1. entails also the pharmaceutically acceptable salts of Pregabalin.
3. The Pregabalin of claim 1. has a median particle size of less than 200 μm.
4. The pharmaceutical excipient of claim 1. consists of, but is not limited to, one or more disintegrant, lubricant, filler and glidant.
5. The disintegrant of claim 4. consists of, but is not limited to microcrystalline cellulose, sodium alginate, Croscarmellose Sodium, croscarmellose sodium, crospovidone and any “super-disintegrants”.
6. The filler of claim 4. consists of, but is not limited to cellulose, lactose, mannitol, starch, sucrose, and calcium phosphate.
7. The lubricant of claim 4. consists of, but is not limited to calcium stearate, magnesium stearate, stearic acid, sodium lauryl sulphate, and magnesium lauryl sulphate.
8. The glidant of claim 4. consists of, but is not limited to silica derivatives, talc, and corn starch.
9. The immediate-release tablet of claim 1. entails the tablet form and the film-coated tablet form. In the case of film-coated tablet form, the percentage weight of Pregabalin refers to the weight of the API in its entirety of the pharmaceutical form (which includes the film-coating).
10. The composition of claim 1. is for the dosage range of 12.5 mg to 600 mg Pregabalin.

The method of preparation of the composition of claim 1. entails the direct compression and the wet granulation methods.

Further embodiments of the invention are enumerated in the following:
1. An immediate-release oral pharmaceutical composition comprising by weight of ≥50.1% Pregabalin, ≥8.0% polyvinylpyrrolidone, ≥3.0% croscarmellose sodium, and from 0% to 38.9% additional pharmaceutical excipient.

In preferred embodiments, the immediate-release oral pharmaceutical composition does not comprises further active agents, other than Pregabalin, meaning that it comprises e.g. by weight of ≥50.1% (or another percentage or a specific dosage, etc., as detailed herein, mutatis mutandis) of an active agent, wherein said active agent is Pregabalin.
2. Immediate-release oral pharmaceutical composition according to item 1, comprising by weight ≥70.1% Pregabalin, preferably ≥80.1% Pregabalin.

In another embodiment, the amount of Pregabalin in the immediate-release oral pharmaceutical composition is ≥87%, more preferably about 89%. In certain embodiments, the amount of Pregabalin in the immediate-release oral pharmaceutical composition is from 50% or 50.1% to 89% or 90% Pregabalin.
3. The Immediate-release oral pharmaceutical composition according to any one of items 1 or 2, wherein the Pregabalin has a median particle size of less than 200 μm.
4. The Immediate-release oral pharmaceutical composition according to any one of items 1 to 3, wherein the additional pharmaceutical excipient comprises one or more components selected from the group comprising disintegrant, lubricant, filler and glidant.
5. The Immediate-release oral pharmaceutical composition according to item 4, wherein the disintegrant is one or more disintegrants selected from the group comprising microcrystalline cellulose, carboxymethyl cellulose, alginic acid, sodium alginate and their derivatives, sodium starch glycolate (such as Glycolys®, Explotab®, Vivastar® P), crospovidone (such as Kollidon®, Kollicoat®) and other super-disintegrants, as well as cellulose, lactose, mannitol, starch and sucrose.
6. The immediate-release oral pharmaceutical composition according to any one of item 4 or 5, wherein the filler is one or more fillers selected from the group comprising cellulose, lactose, mannitol, starch, sucrose, and calcium phosphate, calcium carbonate, maltodextrin, sorbitol and dextrin.

It is known to the skilled person that certain excipients used as fillers can also act as disintegrant, such as cellulose, lactose, mannitol, starch and sucrose. In embodiments where one of these is included as disintegrant, they are not additionally used as fillers, in other words, their inclusion as “disintegrant” or “filler” in the embodiments of the present invention is mutually exclusive.
7. The immediate-release oral pharmaceutical composition according to any one of items 4 to 6, wherein the lubricant is one or more lubricants selected from the group comprising calcium stearate, magnesium stearate, stearic acid, sodium lauryl sulphate, and magnesium lauryl sulphate.
8. The immediate-release oral pharmaceutical composition according to any one of items 4 to 7, wherein the glidant is one or more glidants selected from the group comprising silica derivatives, such as silicone dioxide, talc, and corn starch.
9. The immediate-release oral pharmaceutical composition of any one of items 1 to 8, which is in tablet form or in a film-coated tablet form, wherein in said film-coated tablet form the percentage weight refers to the pharmaceutical composition in its entirety, including the film-coating weight of the tablet core.
10, The immediate-release oral pharmaceutical composition of any one of items 1 to 9, wherein Pregabalin is comprised in a dosage range of 12.5 mg to 600 mg, particularly 25 mg to 300 mg.
11. A method of preparing a tablet comprising a composition according to any one of items 1 to 10, the method comprising
   blending together Pregabalin, polyvinylpyrrolidone and croscarmellose sodium in the ratios according to any one of the preceding items,
   optionally adding additional excipients in the ratio according to any one of the preceding items and blending,
   compressing the blend into tablets,
   optionally adding a film-coating onto the tablets.

1. An immediate-release oral pharmaceutical composition comprising by weight of ≥50.1% Pregabalin, ≥8.0% polyvinylpyrrolidone, ≥3.0% croscarmellose sodium, and from 0% to 38.9% additional pharmaceutical excipient.

2. Immediate-release oral pharmaceutical composition according to claim 1, comprising by weight ≥70.1% Pregabalin, preferably ≥80.1% Pregabalin.

3. The Immediate-release oral pharmaceutical composition according to claim 1, wherein the Pregabalin has a median particle size of less than 200 μm.

4. The Immediate-release oral pharmaceutical composition according to claim 1, wherein the additional pharmaceutical excipient comprises one or more components selected from the group comprising disintegrant, lubricant, filler and glidant.

5. The Immediate-release oral pharmaceutical composition according to claim 4, wherein the disintegrant is one or more disintegrants selected from the group comprising microcrystalline cellulose, carboxymethyl cellulose, alginic acid, sodium alginate and their derivatives, sodium starch glycolate (such as Glycolys®, Explotab®, Vivastar® P), crospovidone (such as Kollidon®, Kollicoat®) and other super-disintegrants, as well as cellulose, lactose, mannitol, starch and sucrose.

6. The immediate-release oral pharmaceutical composition according to claim 4, wherein the filler is one or more fillers selected from the group comprising cellulose, lactose, mannitol, starch, sucrose, and calcium phosphate, calcium carbonate, maltodextrin, sorbitol and dextrin.

7. The immediate-release oral pharmaceutical composition according to claim 4, wherein the lubricant is one or more lubricants selected from the group comprising calcium stearate, magnesium stearate, stearic acid, sodium lauryl sulphate, and magnesium lauryl sulphate.

8. The immediate-release oral pharmaceutical composition according to claim 4, wherein the glidant is one or more glidants selected from the group comprising silica derivatives, such as silicone dioxide, talc, and corn starch.

9. The immediate-release oral pharmaceutical composition of claim 1, which is in tablet form or in a film-coated tablet form, wherein in said film-coated tablet form the percentage weight refers to the pharmaceutical composition in its entirety, including the film-coating weight of the tablet core.

10. The immediate-release oral pharmaceutical composition of claim 1, wherein Pregabalin is comprised in a dosage range of 12.5 mg to 600 mg, particularly 25 mg to 300 mg.

11. A method of preparing a tablet comprising a composition according to claim 1, the method comprising
blending together Pregabalin, polyvinylpyrrolidone and croscarmellose sodium in the ratios according to any one of the preceding claims,
optionally adding additional excipients in the ratio according to any one of the preceding claims and blending,
compressing the blend into tablets,
optionally adding a film-coating onto the tablets.
